(19) United States
(12) Patent Application Publication
(10) Pub. No.: US 2025/0019636 A1
(43) Pub. Date: Jan. 16, 2025

(54) DEVICE FOR SEPARATING MOTILE CELLS

(71) Applicant: MotilityCount ApS, Valby (DK)

(72) Inventors: Jacob Møllenbach LARSEN, Borre (DK); Steen Broch LAURSEN, Odense C (DK)

(21) Appl. No.: 18/708,124

(22) PCT Filed: Nov. 9, 2022
(86) PCT No.: PCT/DK2022/050235

§ 371 Date: May 7, 2024

(30) Foreign Application Priority Data
Nov. 9, 2021 (DK) PA202170546

(51) Int. Cl. C12M 1/26; B01D 61/14; B01D 63/08; B01D 69/02; C12M 1/00; C12N 5/076

(52) U.S. Cl. CPC C12M 33/14; B01D 61/147; B01D 63/08; B01D 69/02; C12M 29/20; C12N 5/061; B01D 2325/0283

(57) ABSTRACT

The present disclosure relates to a mesoscale fluidic device for separating motile cells from non-motile cells, the mesoscale fluidic device including a substrate having a sample application compartment below a medium compartment, and a cell permeable filter with a pore size in the range of 1 μm to 20 μm, the cell permeable filter having an upper surface facing the medium compartment and a lower surface facing the sample application compartment, with the sample application compartment and the medium compartment being in fluid communication via the cell permeable filter, wherein the sample application compartment has a floor and at least one flow structure extending from the floor towards the lower surface of the cell permeable filter facing the sample application compartment. The device is especially useful for obtaining a medium enriched in motile sperm cells from a mammal sperm sample.

### CROSS-REFERENCES TO RELATED APPLICATIONS

This application is a National Stage application of International Patent Application No. PCT/DK2022/050235, filed on Nov. 9, 2022, which claims priority to Danish Patent Application No. PA202170546, filed Nov. 9, 2021, each of which is hereby incorporated by reference in its entirety.

### TECHNICAL FIELD

The present invention relates to a mesoscale fluidic device for separating motile cells from non-motile cells. The mesoscale fluidic device is useful in obtaining a sperm sample enriched in motile cells compared to non-motile cells. The present invention also relates to a method of extracting motile cells from non-motile cells, in particular using the mesoscale fluidic device.

### BACKGROUND

Awareness of decrease in male fertility potential has been high during the last decades, thus creating a demand for having sperm samples analysed and for improving the quality of sperm samples. In particular, it is known that the concentration of motile sperm cells in a sample is the most predictive factor with regard to estimating the fertility of a man (Tomlinson et al., 2013, Human Fertility, 1-19).

The analysis for assessing the number and motility of cells in a sperm sample may be carried out by professionals in a laboratory environment, although home diagnostic devices for estimating male fertility do exist. For example, WO 2014/177157 discloses a system that can estimate the quantity of motile cells in a sample. The device of WO 2014/177157 has a sample application compartment below a cell permeable filter and a conditioning medium compartment above the cell permeable filter, and it was found that by applying a sperm sample below the filter a more efficient separation of motile cells from non-motile cells was provided.

However, while the system of WO 2014/177157 is efficient in quantifying the number of motile cells in a sample, the system has drawbacks due to the limited volume that can be handled, and the system of WO 2014/177157 is not well-suited for separating motile sperm cells from sample volumes larger than 0.5 mL. Furthermore, the physical integrity of the cell permeable filter in the device may be impaired.

It is an object of the invention to provide an improved system for separating motile cells from non-motile cells offering the possibility of extracting the motile cells from larger sample volumes than the prior art. Furthermore, it is an object of the invention to provide a device in which the cell permeable filter does not substantially bulge or stretch when the sample and/or the medium is introduced into the respective compartments. Especially, it is an object to avoid that the cell permeable filter touches the floor or the ceiling of the device, which may inhibit proper functioning of the device.

### SUMMARY

In a first aspect of the present invention relates to a mesoscale fluidic device for separating motile cells from non-motile cells, the mesoscale fluidic device comprising a substrate having a sample application compartment below a medium compartment, and a cell permeable filter with a pore size in the range of 1 μm to 20 μm, the cell permeable filter having an upper surface facing the medium compartment and a lower surface facing the sample application compartment, with the sample application compartment and the medium compartment being in fluid communication via the cell permeable filter, wherein the sample application compartment has a floor and at least one flow structure extending from the floor towards the lower surface of the cell permeable filter facing the sample application compartment.

The sample application compartment of the mesoscale fluidic device may comprise an inlet port and the medium compartment may comprise a pressure relief vent defining a sample flow direction between the inlet port and the pressure relief vent, said pressure relief vent being in gaseous communication with the ambient.

The sample application compartment having a floor may have at least one flow structures with a distance in the interface plane between individual flow structures, between individual flow structures and the perimeters of the sample application compartment, or between the perimeters of the sample application compartment being up to 15 mm, preferably 1 mm to 12 mm. For larger animals, such as horses, it may be advantageous to increase the perimeter above 15 mm to obtain a sufficient sample amount. In an implementation of the invention, the medium compartment comprising a ceiling, an access port, and an pressure relief vent, the pressure relief vent being in gaseous communication with the ambient.

In an implementation of the invention, the sample application compartment comprises an inlet port and the medium compartment comprising a pressure relief vent.

In another implementation of the mesoscale fluidic device the flow structures (25) are walls along or transverse to the sample flow direction(S) or pillars, which walls or pillars extend from the floor of the sample application compartment towards the cell permeable filter.

In a further implementation of the mesoscale fluidic device the inlet port is located at an inlet end of the sample application compartment and the pressure relief vent is located at an opposite or the inlet end of the sample application compartment.

In a further implementation of the mesoscale fluidic device the access port and the pressure relief vent define a medium flow direction between the access port and the pressure relief vent, the medium compartment comprises at least one flow structure.

In a further implementation of the mesoscale fluidic device at least one wall positioned along or transverse to the medium flow direction(S) or at least one pillar, which wall or pillar extend between the ceiling of the medium compartment and the upper surface of the cell permeable filter.

In a further implementation of the mesoscale fluidic device the wall has a shape providing for a meandering flow path of the medium from the access port to the pressure relief vent.

In a further implementation the mesoscale fluidic device the walls or pillars are spaced from each other at a distance (F) in the range of 50 μm to 10 mm.

In another implementation of the mesoscale fluidic device the sample application compartment has a depth between the bottom of the sample application compartment and the lower surface of the cell permeable filter, which depth is in the range of 100 μm to 2 mm. In other implementations, the depth is above 2 mm to accommodate a larger sample amount.

In an implementation of the mesoscale fluidic device the height between the ceiling of the medium compartment and the upper surface of the cell permeable filter is in the range of 50 μm to 2 mm.

In an implementation of the mesoscale fluidic device, the pressure relief vent has a smallest lateral dimension of at least 1 mm and wherein the pressure relief vent is open to the ambient.

In another implementation the mesoscale fluidic device the access port is at an access end of the medium compartment, and the pressure relief vent is at the other end.

In an implementation of the mesoscale fluidic device the mesoscale fluidic device comprises a receiving well in fluid communication with the sample application compartment.

In another aspect, the invention concerns a method for preparing a conditioning medium enriched in motile cells from a sample comprising motile and non-motile cells, comprising the steps of:
providing a mesoscale fluidic device as disclosed above,
applying a conditioning medium to the medium compartment via the access port, the conditioning medium comprising one or more of a nutrient, a salt, a buffer, and/or a viscosity modifying agent,
applying a sample comprising motile cells and non-motile cells to the sample application compartment via the inlet port,
allowing the motile cells to swim through the cell permeable filter to the conditioning medium in the medium compartment in a transition time,
extracting conditioning medium enriched in motile cells from the medium compartment via the access port after the transition time.

The device is especially useful for obtaining motile sperm cells from a sperm sample from a mammal. The device of the invention is therefore especially useful in obtaining cells of high motility from a sample of sperm cells having mixed motility for use in Assisted Reproductive Technologies (ART).

Sperm cells have an inherent preference for swimming towards environments with less density media and letting sperm cells swim up through a filter after being added to a sample chamber below a filter is advantageous to letting sperm cells swim through a filter placed differently as explained in WO 2014/177157. A mesoscale fluidic device for separating motile cells from non-motile cells by letting the cells swim through a filter should preferably have a large surface area to the volume of the chambers adjacent to the two surfaces of the filter in order to increase the amount of cells swimming through the filter. However, when the sample with motile cells is to be added below the filter in a system having a large surface area to volume, e.g. when the height of the sample chamber is at least 10 times smaller than either the width, e.g. the diameter, or the length of the sample chamber, it is generally necessary to push conditioning medium, i.e. to apply a high pressure to the medium when filling the medium chamber, into the medium chamber in order to fill the medium chamber and avoid bubbles in the sample chamber. For example, systems of the prior art have required the sample to be applied the sample chamber using a syringe or the like. Increasing the pressure when applying a sperm sample will create shear forces that can damage the cells and the cell permeable filter may be stretched or bulge, and therefore substantially increased pressures are undesirable.

The sample application compartment comprises one or more flow structures. The flow structures have the main purpose of inhibiting or preventing the filter from stretching or bulging, i.e. preventing or impeding the filter from physical contact with the floor or the ceiling of the device. Another purpose of the one or more flow structures may be to form capillary flow structures allowing the liquids to be drawn into the device by capillary forces. The capillary flow structures are generally defined by the distance between the flow structures in the interface plane, or between the flow structures and the perimeters of the sample application compartment. Thus, the flow structures and the perimeters of the sample application compartment generally determine the space available for liquid in the sample application compartment. It is to be understood that the term “in the interface plane” regarding the flow structures and the distance between individual flow structures means that the positions of the flow structures, e.g. in the floor of the sample application compartment, are extended towards the interface plane. In particular, the flow structures need not be in contact with the cell permeable filter. When the distances between the flow structures, or between the flow structures and the perimeters, is small, e.g. in the range of 50 μm to 1000 μm, liquid will be drawn into the space between the flow structures, or between the flow structures and the perimeters, as appropriate, by capillary forces. Thus, when the distances between the flow structures, or between the flow structures and the perimeters, is small, e.g. in the range of 50 μm to 1000 μm, liquid will be drawn into the space between the flow structures, or between the flow structures and the perimeters, the flow structures may also be referred to as “capillary structures”. However, the present inventor has surprisingly found that when a sperm sample is added to the sample application compartment of the device of the invention, i.e. via the inlet port, the sperm sample will also be drawn into the space between the flow structures, or between the flow structures and the perimeters, even for distances up to, and including, 15 mm. In particular, the distances between the flow structures, or between the flow structures and the perimeters may be in the range of 50 μm to 12 mm. In a specific embodiment, the distances between the flow structures, or between the flow structures and the perimeters are in the range of 50 μm to 1000 μm, e.g. the flow structures are referred to as “capillary structure”. In another embodiment, the distances between the flow structures, or between the flow structures and the perimeters are in the range of 1 mm to 12 mm.

Thus, by suitable design and positioning of the flow structures, the sperm sample will be drawn in the sample flow direction. The present inventor has now found that a sperm sample added at the inlet port is evenly distributed in the sample application compartment without formation of air bubbles when little or no pressure is applied to the sample, in particular via the inlet port, when adding the sample to the sample application compartment, even when the mesoscale fluidic device has a large surface area to the volume of the sample application compartment, e.g. when the height of the sample chamber is at least 10 times smaller than either, in particular both, the width, e.g. the diameter, or the length of the sample application compartment. Surprisingly, it has been realised that the air present in the sample application compartment or the medium compartment can penetrate the pores of the cell permeable filter, when the sample is introduced at the inlet. This is especially relevant when the sample application compartment has a depth between the bottom of the sample application compartment and the lower surface of the cell permeable filter in the range of 100 μm to 2 mm, e.g. of up to 1.5 mm or up to 1 mm. Thereby, a device for providing a sperm sample enriched in motile cells not requiring sample application using substantially increased pressure is provided. Thus, the present invention provides a device for enriching a sperm sample in motile cells where shear induced damage to the sperm cells is limited. Thus, in a preferred embodiment of the method of the invention, the sperm sample is applied to the sample application compartment without increasing the pressure or only increasing the pressure insubstantially due to the escape of the air thorough the pores of the cell permeable filter. For example, the mesoscale fluidic device may comprise a receiving well in fluid communication with the sample application compartment, and the sperm sample is applied to the receiving well, and the sperm sample is drawn into the sample application compartment by capillary forces. The receiving well may for example be in fluid communication with the sample application compartment via a channel having a largest cross-sectional dimension of up to 1 mm. The receiving well is preferably located in an upper surface of the substrate. The receiving well may have a diameter or cross-sectional diameter in the range of 1 mm to 5 mm.

In a specific embodiment, the medium compartment comprises a plurality of walls along the medium flow direction or pillars, which walls or pillars extend between the ceiling of the medium compartment and the upper surface of the cell permeable filter, and which walls or pillars are spaced from each other at a distance in the range of 50 μm to 1000 μm. Thereby, the walls or pillars provide a capillary effect in the medium compartment. Thus, when a conditioning medium is applied to the access port, the conditioning medium will be drawn into the medium compartment via capillary forces. However, in an embodiment, medium compartment does not comprise capillary structures, in particular walls or pillars spaced from each other at a distance in the range of 50 μm to 1000 μm. When capillary structures are present in the medium compartment, the capillary structures may create capillary forces also after motile cells of a sperm sample added to the sample application compartment have crossed the cell permeable filter so that removing motile sperm cells from the medium compartment will require a larger negative relative pressure applied to the liquid containing the motile sperm cells, which may be detrimental to the cells, e.g. by exposing the motile sperm cells to shear forces. Thus, when the medium compartment does not comprise capillary structures, a mesoscale fluidic device for separating motile cells from non-motile cells is provided, which implies a reduced risk of exposing the motile sperm cells to shear forces.

The medium compartment has a pressure relief vent and an access port. The pressure relief vent of the medium compartment is in gaseous communication with the ambient. In particular, since the sample application compartment is in fluid communication with the medium compartment through the filter, the pressure relief vent will provide fluid communication from the sample application compartment to the ambient. The pressure relief vent provide pressure relief so that when a liquid is applied to the inlet port or the access port air can exit the respective compartments via the pressure relief vent. In a specific embodiment, the pressure relief vent is open to the ambient and has a smallest lateral dimension of at least 1 mm. Thus, a pressure relief vent having smallest lateral dimension of at least 1 mm will minimise the risk of motile cells escaping the medium compartment. For example, the pressure relief vent has a smallest lateral dimension in the range of 1 mm to 3 mm. It is also contemplated that the pressure relief vent may have a smallest lateral dimension of less than 1 mm. For example, the pressure relief vent may have a smallest lateral dimension in the range of 0.5 mm to 1 mm.

In use, an appropriate medium, e.g. a conditioning medium with one or more of a nutrient, a salt, a buffer, and/or a viscosity modifying agent, is added via the access port. The conditioning medium preferably has a lower density than the density of the sperm sample in order to induce the motile sperm cells to swim toward the conditioning medium. The medium will flow into the medium compartment, where the pressure relief vent will allow that the medium to replace the air in the medium compartment. A sample with motile cells and non-motile cells, in particular a sperm sample from a mammal, is added to the sample application compartment via the inlet port. It is preferred that the mesoscale fluidic device comprises a receiving well in fluid communication with the sample application compartment, and that the sperm sample is added to the receiving well, and further that the receiving well is in fluid communication with the sample application compartment via a capillary channel so that the sperm sample at least partly is drawn into the sample application compartment with the aid of capillary forces.

After application of the sperm sample, the motile cells are allowed to swim through the cell permeable filter to the medium compartment. The time allowed for the sperm samples to swim through the cell permeable filter, in the context of the invention, is referred to as the transition time. The transition time may be chosen freely but is typically in the range of 1 minute to 60 minutes.

When the transition time has passed, the liquid, i.e. conditioning medium now containing the motile cells, is extracted from the medium compartment via the access port. The access port may be dimensioned to allow an appropriate tool, e.g. any kind of pipette or syringe, to be inserted into the liquid in the medium compartment. In a specific embodiment, the access port is fluidly connected, e.g. via a channel, to an access well. In particular the access well may be located at an upper surface in the substrate. The access well, or optionally the access port preferably may have a smallest cross-sectional dimension in the range of 1 mm to 5 mm. In particular, the access well will generally be larger than the tip of the pipette or syringe. When the access well is larger than the tip of the pipette or syringe, operation of the mesoscale fluidic device is easier than when the tip of the pipette or syringe has the same approximate size as the access well, e.g. the diameter of the access well, it is ensured that the pipette or syringe cannot become stuck in the access well, and in particular, shear damage to the sperm cells is prevented. When the mesoscale fluidic device has an access well, the access well may be in fluid communication with the access port, e.g. via a channel. The channel may have any diameter or cross-sectional dimension.

In an embodiment, the inlet port of the sample application compartment is located at an inlet end of the sample application compartment and the pressure relief vent is located at the other end of the device. The access port may be at an access end of the medium compartment opposite and the pressure relief vent at the other end of the medium compartment. In a preferred embodiment, the inlet port of the sample application compartment and the access port of the medium compartment are located adjacent to each other. A further advantage of the device of the invention is that the flow structures in the sample application compartment provide that a sperm sample added to the sample application compartment via the inlet port can form a uniform distribution, in particular with no air bubbles, in the sample application compartment. This effect is particularly relevant when the distances between the flow structures, or between the flow structures and the perimeters, are in the range of 1 mm to 12 mm. In this case, the sperm sample is drawn into the sample application compartment and distributed evenly, and since the total length of movement of the sperm sample into the sample application compartment may be smaller than when the distances are less than 1 mm, a better concentrating effect is obtained in the mesoscale fluidic device. This is even more relevant when the distances between the flow structures, or between the flow structures and the perimeters, are in the range of 2 mm to 12 mm, such as 3 mm to 12 mm. Thus, the sperm sample fills the sample application compartment in an amount corresponding to the volume of the sperm sample. The medium compartment may be filled with conditioning medium, but due to the laminar flow conditions prevalent in the medium compartment, it is possible to only extract liquid from medium compartment above the section of the sample application compartment into which the sperm sample has been drawn. Thus, when the inlet end and the access ports are located adjacent to each other, it is possible to extract only the volume of liquid from the medium compartment where the sperm cells have migrated into. Thereby, the volume of the liquid enriched in motile sperm cells is kept at a minimum. Thus, a flexible system for enriching motile sperm cells in a sperm sample is provided.

In the context of this invention the terms “motile” and “motility” refer to cells that are capable of moving in a liquid independently of any flow of the liquid. In particular, motile cells are capable of moving in non-flowing liquids. The motile cells may also be said to be “travelling” or “swimming” etc. Motility may be considered to be random, or cells may respond to a stimulus by swimming, e.g. by swimming towards or away from a given condition. In the mesoscale fluidic device, the conditioning medium is preferably of a lower density than the density of a typical sperm sample. When a conditioning medium of a lower density than the density of the sperm sample is added to the medium compartment, motile sperm cells in a sperm sample added to the sample application compartment will preferentially swim toward the medium compartment. Common other stimuli may be for motile cells to move in response to a chemical gradient (“chemotaxis”), a temperature gradient (“thermotaxis”), a light gradient (“phototaxis”), a magnetic field line (“magnetotaxis”), or an electric field (“galvanotaxis”). Relevant stimuli will be known to the skilled person. The cellular motility may be induced by providing a stimulus relevant to motile sperm cells in order to make the cell swim from the sample application compartment to the medium compartment. For example, a chemokine or other chemical may be placed in the medium compartment to attract motile sperm cells added in the sample application compartment. In an embodiment, the medium compartment contains an attractant that will attract sperm cells based on whether they contain an X or a Y chromosome. Such attractants are well-known to the skilled person (see e.g. T. Umehara, N. Tsujita, M. Shimada, PLOS Biology, 13 Aug. 2019, https://doi.org/10.1371/journal.pbio.3000398) and are also known as ovipositional attractants.

In the context of this invention the term “mesoscale” is intended to cover a range of sizes where the smallest dimension of channels is in the range from about 10 μm to about 10 mm, e.g. about 100 μm to about 5 mm, typically about 2 mm, although the channels may also contain constrictions. Likewise, a compartment may be of a depth of about 100 μm to about 20 mm or more, such as about 500 μm to about 2 mm, e.g. about 500 μm or about 1 mm, and the largest horizontal dimension may be from about 1 mm to about 50 mm, e.g. from about 1 mm to about 30 mm or from about 1 mm to about 20 mm, or from about 1 mm to about 10 mm, e.g. from about 2 mm to about 6 mm. In an implementation, such as an implementation for horses, the mesoscale device may have a compartment with a depth of more than 20 mm and/or the largest horizontal dimension may be above 50 mm. It can generally be said that fluids in mesoscale fluidic systems will be flowing under laminar conditions, and fluidic systems with channels or chambers different from those defined above may well be described as “mesoscale” as long as fluids contained in the systems flow under laminar conditions.

A “filter” according to the present invention is to be understood in the broadest terms as a unit capable of separating solids, e.g. cells, and liquid. Thus, the filter may be, e.g. a filter paper, a filter membrane etc., a sieve, a packed bed of particles. The present system comprises a cell permeable filter, which allows cells to traverse the filter. The cell permeable filter has a pore size from 1 μm to 20 μm, e.g. 1 μm to 3 μm, such as 1, 3, 5, 8, 10, 12, 15 μm etc., allowing motile sperm cells to swim through it while at the same time providing a pressure drop across the filter. A preferred pore size is in the range of 8 μm to 10 μm, e.g. about 10 μm. In one embodiment the cell permeable filter is a nucleopore filter. The thickness of the cell permeable filter may be chosen freely. However, in a preferred embodiment the sample application compartment has 4 to 12 pillars as flow structures, which provide support to the cell permeable filter, and the cell permeable filter has a thickness in the range of 10 μm to 25 μm.

The mesoscale fluidic system of the invention is employed with a conditioning medium. The term “conditioning medium” is not intended to be limiting, but “conditioning” refers to that the medium may contain components necessary for analysis of the motile cells and also for keeping the cells viable. Thus, the conditioning medium may contain pH buffers, salts, nutrients as appropriate to a cell type of interest. The conditioning medium may also contain a detection agent, or a detection agent may be added separately to a conditioning medium in the system or present in a dried form in a channel or chamber.

Once a sample with motile cells is added to the sample application compartment, the motile sperm will encounter the cell permeable filter and swim along the encountered surface thereby swimming through the cell permeable filter. After having moved through the cell permeable filter to the medium compartment, the motile sperm cells can be extracted with the liquid via the access port, e.g. using a pipette, a syringe, or the like. Thereby, a liquid enriched in motile cells compared to the sample added to the sample application compartment is provided.

The cell permeable filter has a surface facing the sample application compartment, i.e. the “lower surface of the cell permeable filter”, and a surface facing the medium compartment, i.e. the “upper surface of the cell permeable filter”. In general, the lower surface of the cell permeable filter and the upper surface of the cell permeable filter will be as large as possible relative to the sizes of the respective compartments, and in particular the lower surface of the cell permeable filter and the upper surface of the cell permeable filter may have substantially the same size.

In general, the access port and the pressure relief vent are at opposite ends of the medium compartment. For example, the access port may be at an access end of the medium compartment, and the pressure relief vent may be at an opposite end of the medium compartment. Likewise, the inlet port may be located at an inlet end of the sample application compartment and the pressure relief vent may be located at an opposing end of the device, so that a sample flow direction is defined from the inlet port to the pressure relief vent. In particular, the inlet end and the pressure relief vent may be in opposite locations of the device. In another embodiment, the inlet port for the sample and the pressure relief vent are placed adjacent to each other. Thus, the medium is allowed to fill the medium compartment and the sample is allowed to fill the sample application compartment, while the air in the sample application compartment is allowed to escape through the pores of the filter and subsequently the pressure relief vent.

The flow structures may have any shape desired, and there may be any number of flow structures. For example, the flow structures may be bumps or the like extending from a surface in the sample application compartment. In an embodiment, the flow structures extend along the sample flow direction. The flow structures may be located in any section over the length of the sample flow direction, for example the flow structures may be present over 10% to 100% of the length of the sample flow direction. It is preferred that flow structures are found in up to 50% of the length of the sample flow direction from the outlet.

When the flow structures extend from the floor of the sample application compartment, the extension will typically be in the range 40% to 100% of the distance between the floor of the sample application compartment and the lower surface of the cell permeable filter. The distance between a top surface of the flow structures extending from the floor of the sample application compartment, i.e. the surface facing the lower surface of the cell permeable filter, may be up to 1.2 mm, e.g. in the range of 0 mm to 1 mm, or in the range of 0.2 mm to 0.4 mm, and the distance, when larger than 0 mm, creates the same flow effect as observed between the flow structures or between the flow structures and the perimeters of the sample application compartment in the interface plane, i.e. the sperm sample is drawn into the space between the top surfaces and the lower surface of the cell permeable filter.

In a specific embodiment, the flow structures are walls or pillars extending from the floor of the sample application compartment, e.g. between the floor of the sample application compartment and the lower surface of the cell permeable filter, along the sample flow direction. The flow structures may also be a combination of walls and pillars. When the flow structures are walls, the walls may extend along the sample flow direction. The flow structures, regardless whether they are wall, pillars or a combination of walls and pillars, may be spaced regularly or at irregular intervals.

In an embodiment, the flow structures are pillars extending from the floor of the sample application compartment, for example the pillars may have a cylindrical shape or a frustoconical shape with a diameter in the range of 0.1 mm to 1.0 mm, e.g. in the range of 0.2 mm to 0.6 mm. In a specific embodiment, the sample application compartment contains 4 to 12 pillars as flow structures. The pillars are preferably distributed evenly in the sample application compartment. In the context of the invention, the term “evenly distributed”, when used to the describe the distance between the flow structures, especially pillars, means that a group of flow structures can be defined where the smallest distance between pairs of flow structures in the group varies from the smallest distance between other pairs of flow structures in the group with plus or minus 25% from an average value.

When the sample application compartment has at least 4 pillars as flow structures, especially when the pillars are evenly distributed, and when the extension from the floor of the sample application compartment is at least 80% of the distance between the floor of the sample application compartment and the lower surface facing the sample application compartment, the pillars provides for a suitable structural support to the cell permeable filter. This is particularly relevant when the sample application compartment has at least 6 pillars, or at least 8 pillars, as flow structures. In the context of the invention, “support to the cell permeable filter” means that the sample application compartment cannot collapse due to bending of the cell permeable filter. This provides that manufacture of the mesoscale fluidic device of the invention is simplified since the mesoscale fluidic device is not required to be rigid. Moreover, this further provides that the volume of the sample application compartment is constant, since variations due to bending of the cell permeable filter are limited. In particular, the support provided by the pillars, e.g. 4 to 12 evenly distributed pillars, allows that the sample application compartment can be wide. For example, the width, i.e. the normal distance between the perimeters, of the sample application compartment may be in the range of 25 mm to 100 mm without risk that the cell permeable filter bends and the sample application compartment collapses. Thereby, a large surface area to the volume of the chambers adjacent to the two surfaces of the filter is available. When the width, i.e. the normal distance between the perimeters, of the sample application compartment is in the range of 25 mm to 100 mm, and the sample application compartment has at least 4 evenly distributed pillars, a higher concentrating power is provided in a mesoscale fluidic device for separating motile cells from non-motile cells.

The support furthermore allows that thinner cell permeable filters can be used in the mesoscale fluidic device. Thus, when the sample application compartment contains 4 to 12 evenly distributed pillars as flow structures, the manufacturing costs are lowered. Thus, in a preferred embodiment, the flow structures are pillars, and the number of pillars is in the range of 4 to 8, and the pillars are evenly distributed, e.g. with distances between the pillar, or between the pillars and the perimeters, being in the range of 6 mm to 12 mm. The pillars may, for example, extend from the floor of the sample application compartment at a distance in the range of 0.4 mm to 1.6 mm, e.g. 0.8 mm to 1.2 mm, in particular, the extension from the floor of the sample application compartment is preferably at least 80% of the distance between the floor of the sample application compartment and the lower surface of the filter facing the sample application compartment.

The upper surface of the cell permeable filter can be said to define a bottom of the medium compartment, and the medium compartment will have a ceiling, so that the medium compartment has height between the ceiling and the upper surface of the cell permeable filter. Likewise, the lower surface of the cell permeable filter can be said to define a ceiling of the sample application compartment, which also has a floor, so that the sample application compartment has a depth between the bottom and the lower surface of the cell permeable filter. When the lower surface of the cell permeable filter and the upper surface of the cell permeable filter have substantially the same size, a concentrating effect is provided when the height of the medium compartment is smaller than the depth of the sample application compartment. The motile cells will move from the volume in the sample application compartment to the volume in medium compartment, which is proportionally smaller depending on the difference between the height of the medium compartment and the depth of the sample application compartment. For example, in an embodiment ratio between the depth of the sample application compartment and the height of the medium compartment is in the range of 1.5:1 and 20:1, e.g. 2:1 to 10:1, or 3:1 to 5:1. This is particularly advantageous when the inlet end of the sample application compartment and the access end of the medium compartment are located adjacent to each other, the exit end and the outlet end are also adjacent to each other and opposite the location of the adjacent inlet end and the access end, since only a limited volume, i.e. corresponding to the volume above the sample application compartment into which a sperm sample has been drawn depending on its volume. In another embodiment, the ratio between the depth of the sample application compartment and the height of the medium compartment is approximately 1.

In general, the lower the distance between the bottom or floor of the sample application compartment and the lower surface of the cell permeable filter, the more efficient the separation of the motile cells from non-motile cells. Thus, in an embodiment of the invention, the sample application compartment has a depth between a bottom of the sample application compartment and a lower surface of the cell permeable filter, which depth is in the range of 100 μm to 2 mm.

The substrate may be made from any convenient material, such as a polymer, a glass, a metal, a ceramic material or a combination of these, and channels and chambers in the substrate may be created using any appropriate method. Appropriate methods comprise milling, micromilling, drilling, cutting, laser ablation, hot embossing, injection moulding and microinjection moulding, and 3D printing. For example, the substrate may be made from a thermoplastic polymer, e.g. poly(methyl methacrylate) (PMMA), cyclic olefin copolymer (COC), polycarbonate, polystyrene, polyethylene, polyethylene terephthalate (PET), etc. It is preferred that the substrate material has a hydrophilic surface, and therefore COC and PMMA are preferred materials. However, the skilled person knows how to increase the hydrophilicity of a polymer material for less hydrophilic materials, e.g. hydrophobic materials may be treated with plasma or coated.

The cell permeable filter may be any filter with an appropriate pore size, e.g. 1, 3, 5, 8, 10, 12, 15 μm, etc. For example, the cell permeable filter may be a polycarbonate membrane with pores created by irradiation. Such filters are known under the trademarks nucleopore filter and isopore filter. The thickness of the filter will generally be in the range of 1 μm to 50 μm, e.g. 5 μm to 20 μm, e.g. about 10 μm or about 8 μm. It is further preferred that the cell permeable filter has a hydrophilic surface, e.g. as available with a polycarbonate membrane.

In an embodiment, the substrate comprises two polymer layers of a thermoplastic polymer, into which channels and chambers have been formed, e.g. by injection moulding, and the two polymer layers are joined with a filter between them.

Any embodiment of the invention may be used in any aspect of the invention, and any advantage for a specific embodiment applies equally when an embodiment is used in a specific aspect.

### BRIEF DESCRIPTION OF THE DRAWINGS

In the following the invention will be explained in greater detail with the aid of an example and with reference to the schematic drawings, in which

FIG. 1 shows a schematic cross-sectional drawing of the mesoscale fluidic device of the invention.

FIG. 2 shows a top-view of the sample application compartment of an embodiment of the mesoscale fluidic device of the invention.

FIG. 3 shows a top-view of the sample application compartment of an embodiment of the mesoscale fluidic device of the invention.

FIG. 4 shows top-views of the mesoscale fluidic device of the invention and the sample application compartment.

FIG. 5 shows an exploded view of an embodiment of the device. Note that the filter has been removed from clarity reasons.

FIG. 6 discloses the same embodiment as in FIG. 5. However, the figure has been tilted for showing the underside of the upper substrate layer and the lower face of the lower substrate layer.

FIG. 7 shows an exploded view of an embodiment of the device. Note that the filter has been removed from clarity reasons.

FIG. 8 discloses the same embodiment as in FIG. 7. However, the figure has been tilted for showing the underside of the upper substrate layer and the lower face of the lower substrate layer.

### DETAILED DESCRIPTION

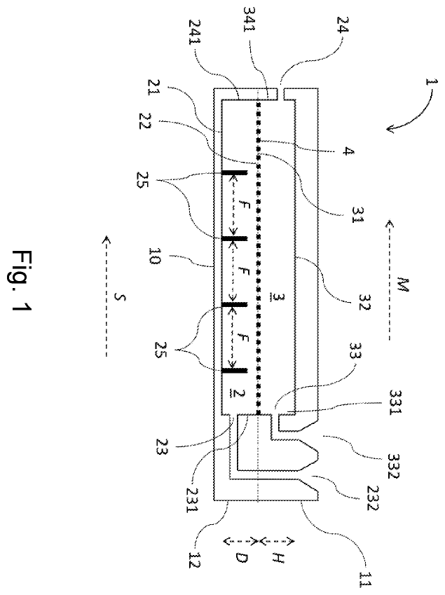

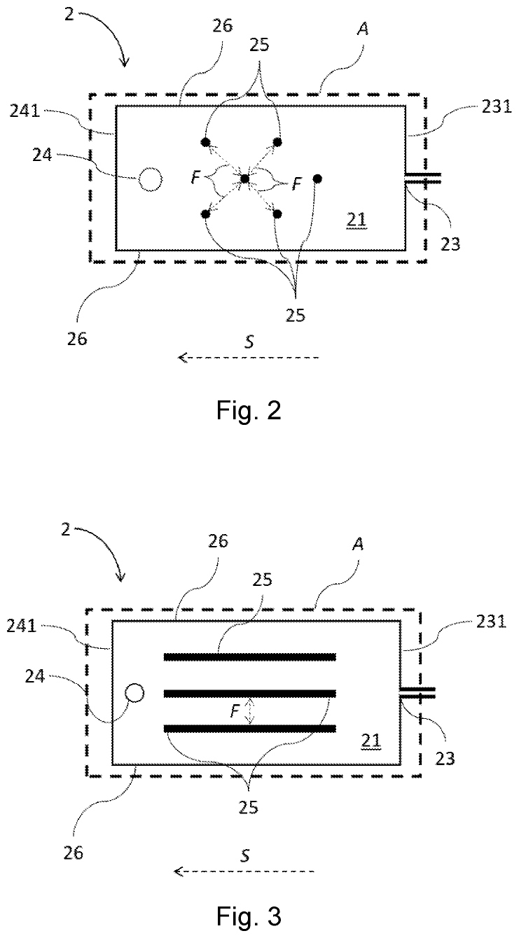

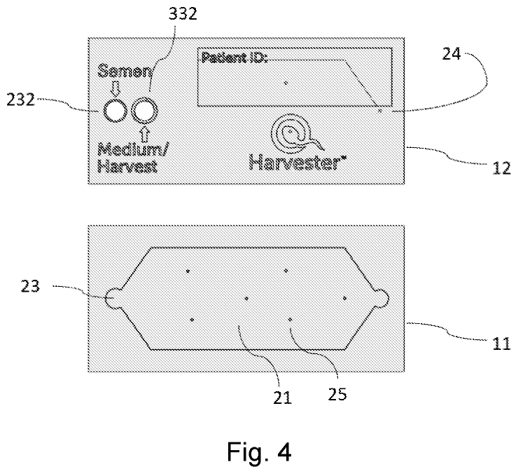

The present invention relates to a mesoscale fluidic device 1 for separating motile cells from non-motile cells and to a method of separating motile cells from non-motile cells. A schematic cross-sectional side view of an embodiment of the mesoscale fluidic device 1 is illustrated in FIG. 1, and top views of two embodiments of the sample application compartment 2 of the mesoscale fluidic device 1 are illustrated in FIG. 2 and FIG. 3. FIG. 4 shows the upper substrate layer 11 (upper panel) and a lower substrate layer 12 (lower panel) of an embodiment of the mesoscale fluidic device 1. The Figures are not drawn to scale. The mesoscale fluidic device 1 comprises a substrate 10 having two substrate layers, i.e. an upper substrate layer 11 and a lower substrate layer 12, of a thermoplastic polymer, e.g. poly(methyl methacrylate) (PMMA), injection moulded to contain the sample application compartment 2 and the medium compartment 3 and channels and other structures as appropriate. In FIG. 1, the mesoscale fluidic device 1 is shown with a dashed line separating the upper substrate layer 11 and the lower substrate layer 12, however this dashed line is only for illustration and not part of the mesoscale fluidic device 1. The substrate layers 11,12 have sizes of 25 mm×50 mm and thicknesses of 2 mm.

The substrate layers 11,12 are welded ultrasonically or glued to a nucleopore filter of 10 μm thickness and having a pore size of 10 μm or 8 μm to provide the cell permeable filter 4 and thereby form the sample application compartment 2 and the medium compartment 3. The cell permeable filter 4 has an upper surface 31 facing the medium compartment 3 and a lower surface 22 facing the sample application compartment 2. The distance from the lower surface 22 to the floor 21 of the sample application compartment 2 defines the height H of the sample application compartment 2, and the distance from the upper surface 31 to the ceiling 32 the medium compartment 3 defines the depth D of the medium compartment 3. The sample application compartment 2 and the medium compartment 3 are thus formed in the surfaces of the substrate layers 11,12 to have a depth D and a height H, respectively, from the surface of 1 mm or 1.5 mm. The perimeters 26 of the sample application compartment 2 define the sample application compartment 2 to have a width of 20 mm and a length of 40 mm. The medium compartment 3 also has a width of 20 mm and a length of 40 mm.

The cell permeable filter 4 defines an interface plane A, which is illustrated with a box drawn as a dashed line in FIG. 2 and FIG. 3. The interface plane A is thus defined between the sample application compartment 2 and the medium compartment 3. In the embodiments of FIG. 2 and FIG. 3, the flow structures 25 extend from the floor 21 of the sample application compartment 2 toward the cell permeable filter 4, so that FIG. 2 and FIG. 3 illustrate the projections of the flow structures 25 on the interface plane A. In the embodiments of FIG. 2 and FIG. 3, the sample application compartment 2 and the medium compartment 3 have the same size, so that the full surface areas of the respective compartments are available for sperm cells to cross the cell permeable filter 4.

The medium compartment 3 has an access point 33 at the access end 331 and an pressure relief vent 24 at the other end 341 opposite the access end 331, and the sample application compartment 2 has an inlet port 23 at the inlet end 231. The inlet end 231 and the access end 331 are at the same location in the mesoscale fluidic device 1.

The inlet port 23 comprises a channel of 500 μm diameter in the lower substrate layer 11, which is in fluid communication with the receiving well 232 in the top surface of the upper substrate layer 12. The access port 33 comprises a channel of 500 μm diameter in the upper substrate layer 12, which is in fluid communication with an access well 332 in the top surface of the upper substrate layer 12. The receiving well 232 is adjacent to the access well 332. The inlet port 23 and the pressure relief vent 24 define a sample flow direction S between the inlet port 23 and the pressure relief vent 24, and the access port 33 and the pressure relief vent 24 define a medium flow direction M between the access port 33 and the outlet 34. The filter has pores of a size allowing air to escape from sample compartments 2 when filled with semen. The air penetrading the filter pores is allowed to pass through the pressure release vent 24 to balance the ambient pressure and the pressure inside the device.

The pressure relief vent 24 comprises a channel of 500 μm diameter in the upper substrate layer 12. The pressure relief vent 24 is shown in the end wall of the upper substrate layer 12, although it may also be present in the ceiling of the medium compartment 3 so that the pressure relief vent 24 can be in the top surface of the upper substrate layer 12. The access port 33 may be used as an inlet as well as an outlet. Thus, the cell medium may be supplied through the access port 33 and after the semen cells have been allowed sufficient time to penetrate the filter 4 they may be subsequently extracted from the access port.

In the embodiment of FIG. 2 the sample application compartment 2 contains six frustoconical pillars as flow structures 25 having a height of 90% of the depth of the sample application compartment 2, e.g. 0.9 mm or 1.35 mm, respectively, width a width at the base of 0.4 mm. The pillars 25 are distributed evenly in the sample application compartment 2 to have the distance F between the flow structures 25. FIG. 2 shows the pressure relief vent 24. It is to be understood that the pressure relief vent 24 is above the floor 21 of the sample application compartment 2. The distance F between the flow structures is 8 mm.

In the embodiment of FIG. 3, the sample application compartment 2 contains three walls as flow structures 25 having a height of 90% of the depth of the sample application compartment 2, e.g. 0.9 mm or 1.35 mm, respectively. The walls 25 has a length of 80% of the distance between the inlet port 23 and the pressure relief vent 24. FIG. 3 shows the pressure relief vent 24. It is to be understood that the pressure relief vent 24 is above the floor 21 of the sample application compartment 2.

FIG. 4 discloses the upper substrate layer 11 and a lower substrate layer 12 of the mesoscale fluid device of a specific embodiment. The filter has been left out for clarity reasons. The upper surface of the upper substrate layer is provided with a receiving well 232 for the inlet of semen and a access well 332 for supply of conditioning medium and extraction of the conditioning medium enriched in sperm cells after a certain incubation period. Also visible on the drawing is the pressure relief vent 24 allowing air to escape from the interior of the device when conditioning medium and/or semen is introduced into the device 1.

The upper part of the lower substrate layer 11 is shown in the lower panel of FIG. 4. The drawing shows pillars 25 position at the floor 21. The pillars are extending upwards from the floor and are adapted for supporting the cell permeable filter. An inlet port 23 is provided at the left-handed side of the drawing and communicate liquidly with the receiving well 232. When in use, the semen sample is introduced at the receiving well 232 and the medium is introduced at the access well 332. During a certain incubation time, the cells are allowed to cross the filter from the sample application compartment 2 to the medium compartment. When the incubation period is finished, the medium enriched in cells may be extracted through the access port 332.

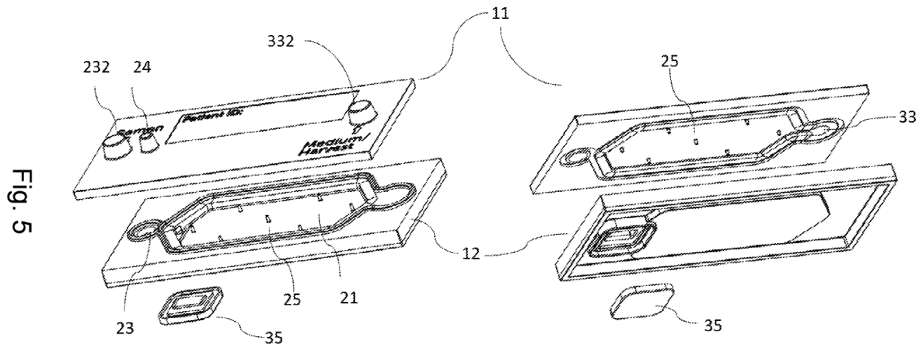

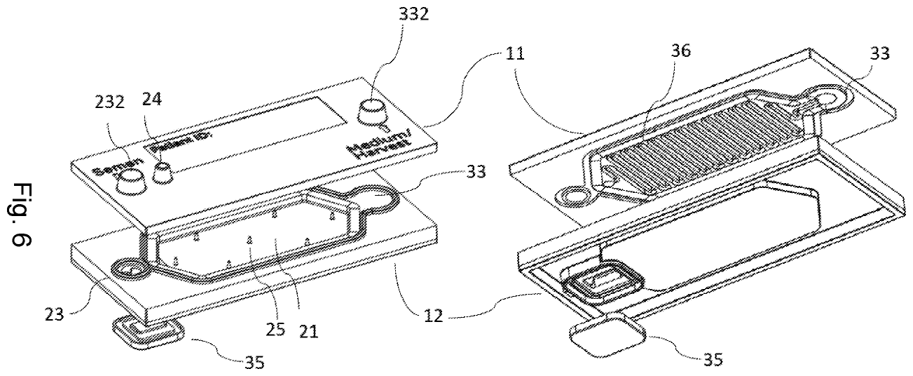

FIGS. 5 and 6 disclose the same embodiment of the mesoscale fluidic device 1 in which the right panel is slightly tilted relative to the mesoscale fluidic decide 1 in the left panel for exposure of the lower part of the upper substrate layer 11. The embodiment comprises 4 parts, i.e. the upper substrate layer 11, the cell permeable filter 4, the lower substrate layer 12, and the lid 35. The upper substrate layer comprises a receiving well 232 for the introduction of the semen sample, an access port 33 for delivering a medium and subsequent harvesting of the medium enriched in the semen cells, and a pressure relief vent 24 allowing air from the interior of the mesoscale fluidic device 1 to escape when the semen sample and/or the medium is introduced. On the lower side of the upper substrate layer flow structure in the shape of pillars are provided. The pillars extents from the ceiling 32 into the interior of the mesoscale fluidic device 1. The cell permeable filter is positioned between the upper and the lower substrate layer. However, the cell permeable filter 4 is not included in the drawing to increase the clarity.

The lower substrate layer 12 comprises an inlet port 23 communicating liquidly with the receiving well 232. The inlet allows the sample to flow to the sample application compartment 2, i.e. the space between the floor 21, the filter 4 and the perimeter of the compartment. The floor 21 of the lower substrate layer 12 comprises flow structures 25 in the form of pillars. The pillars 25 of the lower substrate layer are positioned opposite the pillars of the upper substrate layer 11 to enable support of the filter from both sides. The support from each side provides for a filter that do not substantially bulge or stretch when the sample and/or the medium is introduced into the respective compartments. During the incubation, cells are allowed to move through the filter to enrich the medium in cells. The medium enriched in cells are extracted through the access port 33.

The lid 35 when positioned in the corresponding depression in the lower part of the lower substrate layer participates in the formation of the inlet port 23.

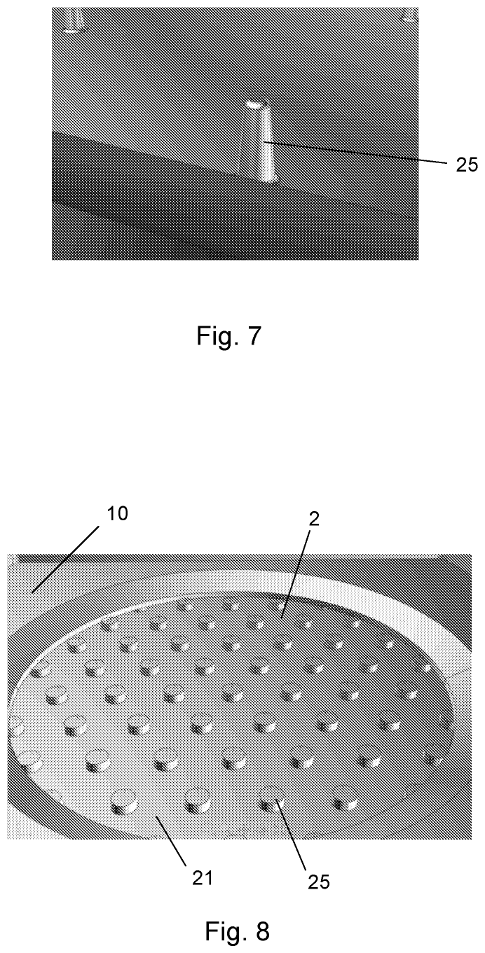

FIG. 7 and FIG. 8 disclose the same embodiment of the mesoscale fluidic device 1 in which the right panel is slightly tilted relative to the mesoscale fluidic decide 1 in the left panel for exposure of the lower part of the upper substrate layer 11. The main difference between the former embodiment illustrated in FIG. 5 and FIG. 6 is the presence a meandering flow channel 36 in the lower part of the upper substrate layer 11. The meandering flow channel 36 extends from the access port 33 for the medium and the pressure relief vent 24. The meandering flow path is formed by sets of flow structures in the shape of walls extending from an alternating perimeter to close to the opposite perimeter. The set of flow structures alternates in the sense that a first flow structure 25 extends from a first perimeter and a neighbouring second flow structure extends from the opposite perimeter. By repeating the set of flow structures the meandering flow path is formed as the space between the walls. The walls may be rounded at the base of the walls to create a flow path without sharp corners.

The lower substrate layer 12 comprises pillars 25 positioned opposite the walls in the lower part of the upper substrate layer to support the cell permeable filter from both sides.

### Example 1

The mesoscale fluidic devices 1 described above and having depths D and heights H of 1.5 mm and 1 mm and having cell permeable filters 4 with pore sizes of 10 μm and 8 μm were tested with sperm samples. Sperm samples were provided and initially analysed for the number of motile cells. A conditioning medium was added to the access wells 332 of each of the mesoscale fluidic devices 1 to fill the medium compartment 3 with conditioning medium. The sperm samples were then added to the receiving wells 232 of the mesoscale fluidic devices 1, and the sperm cells were allowed to migrate from the sample application compartment 2 to the medium compartment 3. After incubation, the liquids in the medium compartments 3 were extracted via the access wells 332 to provide samples enriched in motile sperm cells, and the enriched samples were analysed for motile cells sperm cells and non-motile sperm cells.

The percentages of motile cells in the added samples and in the treated samples and the recovery in the treated samples are shown in Table 1 for mesoscale fluidic devices 1 with H and D of 1.5 mm and Table 2 for mesoscale fluidic devices 1 with H and D of 1.0 mm. In Table 1, sample 1 was allowed an incubation time of 35 minutes, sample 2 was allowed an incubation time of 1 hour, and samples 3 to 5 were allowed an incubation time of 30 minutes. In Table 2, the incubation time was 30 minutes.

TABLE 1

| | Raw | | | | |
|---|---|---|---|---|---|
| | sample | 8 μm pore size | | 10 μm pore size | |
| Sample | % motile | % motile | % recovery | % motile | % recovery |
| 1 | 63.4 | 100.0 | 35.6 | 92.0 | 51.1 |
| 2 | 71.0 | 100.0 | 47.7 | 100.0 | 47.7 |
| 3 | 55.0 | | | 95.0 | 57.0 |
| 4 | 55.0 | | | 99.0 | 65.0 |
| 5 | 55.0 | 99.0 | 48.0 | | |

TABLE 2

| | Raw samples | 10 μm pore size | |
|---|---|---|---|
| Sample number | % motile | % motile | % recovery |
| 1 | 71.4 | 98.8 | 32.0 |
| 2 | 42.9 | 95.8 | 38.3 |

Thus, the mesoscale fluidic devices 1 of the invention consistently provided enrichment to above 90% in motile sperm cells.

While the present disclosure has been illustrated and described with respect to a particular embodiment thereof, it should be appreciated by those of ordinary skill in the art that various modifications to this disclosure may be made without departing from the spirit and scope of the present disclosure.

1. A mesoscale fluidic device for separating motile cells from non-motile cells, the mesoscale fluidic device comprising a substrate having a sample application compartment below a medium compartment, and a cell permeable filter with a pore size in the range of 1 μm to 20 μm, the cell permeable filter having an upper surface facing the medium compartment and a lower surface facing the sample application compartment, with the sample application compartment and the medium compartment being in fluid communication via the cell permeable filter,
wherein the sample application compartment has a floor and at least one flow structure extending from the floor towards the lower surface of the cell permeable filter facing the sample application compartment.

2. The mesoscale fluidic device according to claim 1, wherein a distance (F) in the interface plane (A), defined by the cell permeable filter, between individual flow structures and the perimeters of the sample application compartment is up to 15 mm.

3. The mesoscale fluidic device according to claim 1, wherein the medium compartment comprising a ceiling, an access port, and a pressure relief vent, the pressure relief vent being in gaseous communication with the ambient.

4. The mesoscale fluid device according to claim 1, wherein the sample application compartment comprises an inlet port and the medium compartment comprising a pressure relief vent.

5. The mesoscale fluidic device according to claim 1, wherein the flow structures are walls along or transverse to the sample flow direction(S) or pillars, which walls or pillars extend from the floor of the sample application compartment towards the cell permeable filter.

6. The mesoscale fluidic device according to claim 1, wherein the inlet port is located at an inlet end of the sample application compartment and the pressure relief vent is located at an opposite or the inlet end of the sample application compartment.

7. The mesoscale fluidic device according to claim 1, wherein the access port and the pressure relief vent define a medium flow direction (M) between the access port and the pressure relief vent, the medium compartment comprises at least one flow structure.

8. The mesoscale fluidic device according to claim 7, wherein at least one wall positioned along transverse to the medium flow direction(S) or at least one pillar, which wall or pillar extend between the ceiling of the medium compartment and the upper surface of the cell permeable filter.

9. The mesoscale fluidic device according to claim 1, wherein the wall has a shape providing for a meandering flow path of the medium from the access port to the pressure relief vent.

10. A method for preparing a conditioning medium enriched in motile cells from a sample comprising motile and non-motile cells, comprising the steps of:
providing a mesoscale fluidic device according to claim 1,
applying a conditioning medium to the medium compartment via the access port, the conditioning medium comprising one or more of a nutrient, a salt, a buffer, and/or a viscosity modifying agent,
applying a sample comprising motile cells and non-motile cells to the sample application compartment via the inlet port,
allowing the motile cells to swim through the cell permeable filter to the conditioning medium in the medium compartment in a transition time,
extracting conditioning medium enriched in motile cells from the medium compartment via the access port after the transition time.

11. The mesoscale fluidic device according to claim 2, wherein the medium compartment comprising a ceiling, an access port, and a pressure relief vent, the pressure relief vent being in gaseous communication with the ambient.

12. The mesoscale fluid device according to claim 2, wherein the sample application compartment comprises an inlet port and the medium compartment comprising a pressure relief vent.

13. The mesoscale fluid device according to claim 3, wherein the sample application compartment comprises an inlet port and the medium compartment comprising a pressure relief vent.

14. The mesoscale fluidic device according to claim 2, wherein the wall has a shape providing for a meandering flow path of the medium from the access port to the pressure relief vent.

15. The mesoscale fluidic device according to claim 3, wherein the wall has a shape providing for a meandering flow path of the medium from the access port to the pressure relief vent.

16. The mesoscale fluidic device according to claim 4, wherein the wall has a shape providing for a meandering flow path of the medium from the access port to the pressure relief vent.

17. The mesoscale fluid device according to claim 5, wherein the sample application compartment comprises an inlet port and the medium compartment comprising a pressure relief vent.

18. The mesoscale fluidic device according to claim 6, wherein the wall has a shape providing for a meandering flow path of the medium from the access port to the pressure relief vent.

19. The mesoscale fluidic device according to claim 7, wherein the wall has a shape providing for a meandering flow path of the medium from the access port to the pressure relief vent.

20. The mesoscale fluidic device according to claim 8, wherein the wall has a shape providing for a meandering flow path of the medium from the access port to the pressure relief vent.
